(19) United States
(12) Patent Application Publication
(10) Pub. No.: US 2024/0110932 A1
(43) Pub. Date: Apr. 4, 2024

(54) UNIVERSAL CALIBRATION FOR QUANTITATIVE MASS SPECTROMETRY

(71) Applicant: Roche Diagnostics Operations, Inc., Indianapolis, IN (US)

(72) Inventors: Johannes Pflieger, Holzkirchen (DE); Nicole Pirkl, Penzberg (DE); Martin Rempt, Penzberg (DE); Christoph Zuth, Eglfing (DE)

(21) Appl. No.: 18/541,624

(22) Filed: Dec. 15, 2023

Related U.S. Application Data
(63) Continuation of application No. PCT/EP2022/066362, filed on Jun. 15, 2022.

(30) Foreign Application Priority Data
Jun. 18, 2021 (EP) 21180368.9

(51) Int. Cl. G01N 33/74; G01N 30/72; G01N 33/68; H01J 49/00

(52) U.S. Cl. CPC G01N 33/743; G01N 30/7233; G01N 33/6848; H01J 49/0009; G01N 2030/027; G01N 2333/575; G01N 2496/00

(57) ABSTRACT

The present invention relates to a method of determining an analyte in a sample by mass spectrometry (MS), the method comprising (a) admixing a pre-determined amount of internal calibrator to said sample, wherein said internal calibrator comprises at least two non-identical isotopologues of the analyte at predetermined amounts; (b) determining MS signals of ions generated from said analyte (analyte signal) and from said at least two isotopologues (isotopologue signals); (c) providing a calibration based on the analyte signal and the isotopologue signals determined in step (b); and (d) determining said analyte based on the calibration provided in step (c). Further, the present invention relates to devices, systems, and uses related thereto.

### TECHNICAL FIELD

The present invention relates to a method of determining an analyte in a sample by mass spectrometry (MS), the method comprising (a) admixing a pre-determined amount of internal calibrator to said sample, wherein said internal calibrator comprises at least two non-identical isotopologues of the analyte at predetermined amounts; (b) determining MS signals of ions generated from said analyte (analyte signal) and from said at least two isotopologues (isotopologue signals); (c) providing a calibration based on the analyte signal and the isotopologue signals determined in step (b); and (d) determining said analyte based on the calibration provided in step (c). Further, the present invention relates to devices, systems, and uses related thereto.

### BACKGROUND ART

The quality of absolute quantitation by analytical methods such as mass spectrometry (MS) relies heavily on the quality of the calibration process used to convert measured signals into analyte concentrations. MS signals are in principle suited for external as well as for internal calibration methods. In e.g. liquid chromatography mass spectrometry (LC-MS), matrix effects may be possible to avoid by using suitable LC methods capable to remove in best case all matrix effects from the analyte of interest. If matrix is co-eluted with the analyte signal, only constant matrix effects can be compensated for by external calibration. If the sample matrix is changing (e.g. caused by different sample types or different ionization properties), external calibration is not very accurate.

Nonetheless, the state of the art methodology for MS quantitation relies on the concept of stable isotope dilutions and an external calibration curve generated from reference standards by using signal response (analyte signal/internal standard signal). By increasing calibration frequency, data quality and confidence may be improved, however this increases measuring times and, therefore, costs. Also, while external calibration is widely adapted in the LC-MS environment for batch modus assays, it is not very suited to handle a dynamic, multiplexing system capable of running many different assays in random access. The calibration demand in this case will be too high to allow for regular re-calibrations in this case.

To avoid the aforesaid disadvantages, in particular matrix effects, standard addition methods have been devised in analytical chemistry, in which a standard is added at various concentrations to samples, thereby ensuring that potential matrix effects affect the standard as much as the analyte.

A different approach is the use of an internal standard, which compensates for matrix effects, however requires an additional standard curve to calibrate the analyte/internal standard ratio. All of the aforesaid methods are calibration methods known in the art and described in standard text books.

As a further method, multipoint internal standard regression was described, e.g. in Hoffman et al. (2020), Clin Chem 66(3):474, and in WO 2017/178453 A1. As with external calibration, this method, however, requires that the concentration of the analyte lies within the range fixed by the concentration range of the calibrators used.

### Problem to be Solved

In view of the above, there is still a need in the art for improved means and methods for determining analytes by MS.

### SUMMARY

This problem is addressed by the method, device, system, and use with the features of the independent claims. Advantageous embodiments which might be realized in an isolated fashion or in any arbitrary combinations are listed in the dependent claims.

In accordance, the present invention relates to a method of determining an analyte in a sample by mass spectrometry (MS), the method comprising
(a) admixing a pre-determined amount of internal calibrator to said sample, wherein said internal calibrator comprises at least two non-identical isotopologues of the analyte at predetermined amounts;
(b) determining MS signals of ions generated from said analyte (analyte signal) and from said at least two isotopologues (isotopologue signals);
(c) providing a calibration based on the analyte signal and the isotopologue signals determined in step (b); and
(d) determining said analyte based on the calibration provided in step (c).

In general, terms used herein are to be given their ordinary and customary meaning to a person of ordinary skill in the art and, unless indicated otherwise, are not to be limited to a special or customized meaning. As used in the following, the terms “have”, “comprise” or “include” or any arbitrary grammatical variations thereof are used in a non-exclusive way. Thus, these terms may both refer to a situation in which, besides the feature introduced by these terms, no further features are present in the entity described in this context and to a situation in which one or more further features are present. As an example, the expressions “A has B”, “A comprises B” and “A includes B” may both refer to a situation in which, besides B, no other element is present in A (i.e. a situation in which A solely and exclusively consists of B) and to a situation in which, besides B, one or more further elements are present in entity A, such as element C, elements C and D or even further elements. Also, as is understood by the skilled person, the expressions “comprising a” and “comprising an” in an embodiment refer to “comprising one or more”, i.e. are equivalent to “comprising at least one”. In accordance, expressions relating to one item of a plurality, unless otherwise indicated, in an embodiment relate to at least one such item, in a further embodiment a plurality thereof; thus, e.g. identifying “a cell” relates to identifying at least one cell, in an embodiment to identifying a multitude of cells.

Further, as used in the following, the terms “preferably”, “more preferably”, “most preferably”, “particularly”, “more particularly”, “specifically”, “more specifically” or similar terms are used in conjunction with optional features, without restricting further possibilities. Thus, features introduced by these terms are optional features and are not intended to restrict the scope of the claims in any way. The invention may, as the skilled person will recognize, be performed by using alternative features. Similarly, features introduced by “in an embodiment” or similar expressions are intended to be optional features, without any restriction regarding further embodiments of the invention, without any restrictions regarding the scope of the invention and without any restriction regarding the possibility of combining the features introduced in such way with other optional or non-optional features of the invention.

The methods specified herein below, in an embodiment, are in vitro methods. The method steps may, in principle, be performed in any arbitrary sequence deemed suitable by the skilled person, but in an embodiment are performed in the indicated sequence; also, one or more, in an embodiment all, of said steps may be assisted or performed by automated equipment. Moreover, the methods may comprise steps in addition to those explicitly mentioned above.

As used herein, the term “standard conditions”, if not otherwise noted, relates to IUPAC standard ambient temperature and pressure (SATP) conditions, i.e. in an embodiment, a temperature of 25° C. and an absolute pressure of 100 kPa; also in an embodiment, standard conditions include a pH of 7. Moreover, if not otherwise indicated, the term “about” relates to the indicated value with the commonly accepted technical precision in the relevant field, in an embodiment relates to the indicated value±20%, in a further embodiment ±10%, in a further embodiment ±5%. Further, the term “essentially” indicates that deviations having influence on the indicated result or use are absent, i.e. potential deviations do not cause the indicated result to deviate by more than ±20%, in a further embodiment ±10%, in a further embodiment ±5%. Thus, “consisting essentially of” means including the components specified but excluding other components except for materials present as impurities, unavoidable materials present as a result of processes used to provide the components, and components added for a purpose other than achieving the technical effect of the invention. For example, a composition defined using the phrase “consisting essentially of” encompasses any known acceptable additive, excipient, diluent, carrier, and the like. In an embodiment, a composition consisting essentially of a set of components will comprise less than 5% by weight, in a further embodiment less than 3% by weight, in a further embodiment less than 1% by weight, in a further embodiment less than 0.1% by weight of non-specified component(s).

As indicated above, the method of determining an analyte specified herein may comprise steps in addition to those specified herein above. Additional steps may in particular relate to providing a sample for step (a), to sample pretreatment as specified herein below, to specific MS steps deemed appropriate by the skilled person, like admixture of desorption matrix, ionization, and the like. Moreover, after step (d), the result of the determination may be output to a user, may be stored in a database, may be used in standard mathematical calculations, and the like.

The term “determining” is understood by the skilled person as referring to settling on, concluding on, or ascertaining a fact and/or data. Thus, in an embodiment, determining is qualitative, semiquantitative, or quantitative determination, in an embodiment relates to a quantitative determination. Qualitative determination may be determining whether the value of a parameter is above a predetermined threshold value, e.g. a detection limit or a physiologically relevant threshold value. Semiquantitative determination is assigning a measured value to a pre-established category, e.g. “low”, “medium”, or “high” concentration. In an embodiment, determining is quantitative, i.e. is determining a value of a quantitative measure of a parameter.

The term “determining an analyte in a sample” is understood by the skilled person. In an embodiment, the term relates to a qualitative, semi-quantitative, or quantitative determination of the amount of analyte in a sample, in an embodiment to a quantitative determination of the amount of analyte in a sample. As used herein, the term “amount” of an analyte relates to any quantitative measure of the analyte, and is equivalent to other corresponding measures such as mass fraction and concentration, which can be calculated from the amount in case sample mass or sample volume is known. Thus, the result of measurement of an analyte in a sample may be expressed in any unit deemed appropriate by the skilled person, including arbitrary units, measures of weight, of mass fraction, of concentration, and the like, or measures derived therefrom, e.g. international units according to a pre-defined definition. Methods for determining the amount of an analyte by MS are, in principle, known to the skilled person.

As indicated above, the term “analyte”, as used herein, relates to any chemical compound or group of compounds which shall be determined in a sample. In an embodiment, the analyte is a macromolecule, i.e. a compound with a molecular mass of more than 2500 u (i.e. more than 2.5 kDa). In a further embodiment, the analyte is a biological macromolecule, in particular a polypeptide, a polynucleotide, a polysaccharide, or a fragment of any of the aforesaid. In an embodiment, the analyte is a small molecule chemical compound, i.e. a compound with a molecular mass of at most 2500 u (2.5 kDa), in an embodiment at most 1.5 kDa, in a further embodiment at most 1 kDa. The analyte may be any chemical compound of interest; in an embodiment the analyte is a chemical compound metabolized by a body of a subject, is a compound administered to a subject in order to induce a change in the subject's metabolism, is a chemical compound of interest in technical process, e.g. an educt, an intermediate, or a product, is a chemical compound of interest in an environmental sample, or the like. In an embodiment, the analyte is a chemical compound metabolized by a body of a subject, in particular of a human subject, or is a compound administered to a subject in order to induce a change in the subject's metabolism. Thus, in an embodiment, the analyte is a drug of abuse or a metabolite thereof, e.g. amphetamine; cocaine; methadone; ethyl glucuronide; ethyl sulfate; an opiate, in particular buprenorphine, 6-monoacatylmorphine, codeine, dihydrocodeine, morphine, morphine-3-glucuronide, and/or tramadol; and/or an opioid, in particular acetylfentanyl, carfentanil, fentanyl, hydrocodone, norfentanyl, oxycodone, and/or oxymorphone. In an embodiment, the analyte is a therapeutic drug, e.g. valproic acid; clonazepam; methotrexate; voriconazole; mycophenolic acid (total); mycophenolic acid-glucuronide; acetaminophen; salicylic acid; theophylline; digoxin; an immuno suppressant drug, in particular cyclosporine, everolimus, sirolimus, and/or tacrolimus; an analgesic, in particular meperidine, normeperidine, tramadol, and/or O-desmethyl-tramadol; an antibiotic, in particular gentamycin, tobramycin, amikacin, vancomycin, piperacilline (tazobactam), meropenem, and/or linezolid; an antieplileptic, in particular phenytoin, valporic acid, free phenytoin, free valproic acid, levetiracetam, carbamazepine, carbamazepine-10,11-epoxide, phenobarbital, primidone, gabapentin, zonisamid, lamotrigine, and/or topiramate. In an embodiment, the analyte is a hormone, in particular cortisol, estradiol, progesterone, testosterone, 17-hydroxyprogesterone, aldosterone, dehydroepiandrosteron (DHEA), dehydroepiandrosterone sulfate (DHEA-S), dihydrotestosterone, and/or cortisone; in an embodiment, the sample is a serum or plasma sample and the analyte is cortisol, DHEA-S, estradiol, progesterone, testosterone, 17-hydroxyprogesterone, aldosterone, DHEA, dihydrotestosterone, and/or cortisone; in an embodiment, the sample is a saliva sample and the analyte is cortisol, estradiol, progesterone, testosterone, 17-hydroxyprogesterone, androstendione, and/or cortisone; in an embodiment, the sample is a urine sample and the analyte is cortisol, aldosterone, and/or cortisone. In an embodiment, the analyte is a vitamin, in an embodiment vitamin D, in particular ergocalciferol (Vitamin D2) and/or cholecalciferol (Vitamin D3) or a derivative thereof, e.g. 25-hydroxy-vitamine-D2, 25-hydroxy-vitamine-D3, 24,25-dihydroxy-vitamine-D2, 24,25-dihydroxy-vitamine-D3, 1,25-dihydroxy-vitamine-D2, and/or 1,25-dihydroxy-vitamine-D3. In a further embodiment, the analyte is a metabolite of a subject. In an embodiment, the analyte comprises at least 10, in an embodiment at least 15, atoms of the same element, in an embodiment carbon atoms. In an embodiment, the analyte is Testosterone and the internal calibrator is a preparation of 2,3,4-¹³C₃-Testosterone.

As used herein, the term “sample”, also referred to as “test sample”, relates in principle to any type of composition of matter; thus, the term may refer, without limitation, to any arbitrary sample such as a biological sample, chemical sample, environmental sample, or any other sample comprising or assumed to comprise an analyte of interest. The sample is, in an embodiment, a sample of a subject, in an embodiment a medical or diagnostic sample. In an embodiment, the sample is a liquid sample, in a further embodiment an aqueous sample. In an embodiment, the test sample is selected from the group consisting of: a physiological fluid, including blood, serum, plasma, saliva, ocular lens fluid, lacrimal fluid, cerebrospinal fluid, sweat, urine, milk, ascites, mucus, synovial fluid, peritoneal fluid, and amniotic fluid; lavage fluid; tissue, cells, or the like. In an embodiment, the sample is a blood, plasma, serum, saliva, or urine sample, in a further embodiment a blood, plasma, or serum sample, in a further embodiment a serum or plasma sample. The sample may, however, also be a natural or industrial liquid, in particular surface or ground water, sewage, industrial wastewater, processing fluid, soil eluates, and the like. In an embodiment, the sample comprises or is suspected to comprise at least one chemical compound of interest, i.e. a chemical compound which shall be determined, which is referred to as “analyte”. The sample may comprise one or more further chemical compounds, which are not to be determined and which are commonly referred to as “matrix”. Thus, the sample in an embodiment is a complex matrix sample, in an embodiment comprising more than 100, in a further embodiment more than 1000, chemical compounds. The sample may be used directly as obtained from the respective source or may be subjected to one or more pretreatment and/or a sample preparation step(s). Thus, the sample may be pretreated by physical and/or chemical methods, in an embodiment by centrifugation, filtration, mixing, homogenization, chromatography, precipitation, dilution, concentration, contacting with a binding and/or detection reagent, and/or any other method deemed appropriate by the skilled person.

The term “mass spectrometry”, abbreviated as “MS”, is understood by the skilled person. In an embodiment, the term relates to an analytic technique comprising determining the mass-to-charge ratio of ions generated from an analyte. Thus, an “MS device”, in an embodiment, comprises at least one mass spectrometry unit (MS unit). As used herein, the term “mass spectrometry unit”, in an embodiment, relates to a mass analyzer configured for detecting at least one analyte based on a mass to charge (m/z) ratio of ions of the analyte or a fragments thereof. In an embodiment, the MS unit is a tandem mass spectrometry (MS/MS) unit, in a further embodiment a triple quadrupole MS (QqQ-MS), in a further embodiment in Multiple Reaction Monitoring (MRM) mode. The MS unit comprises at least one detector, typically recording a charge or current induced by, and in an embodiment proportional to, the amount of ions passing through the mass analyzer. The MS device typically further comprises at least one ionization source configured for generating molecular ions and for transferring the molecular ions into the gas phase. Ionization methods and appropriate ionization units are known in the art and include in particular electron ionization (EI), chemical ionization (CI), electrospray ionization (ESI), atmospheric pressure ionization (APCI), atmospheric pressure photoionization (APPI), and matrix assisted laser desorption/ionization (MALDI).

In an embodiment, the MS is a chromatography MS, in particular a gas chromatography MS (GC-MS) or a liquid chromatography MS (LC-MS), terms understood by the skilled person. Thus, in an embodiment, the MS device is configured for performing a combination of chromatography (e.g. LC or GC) with mass spectrometry (MS). Thus, the device, in an embodiment, comprises at least one LC and/or GC unit, and at least one MS unit, wherein the LC and/or GC unit(s) and the MS unit are coupled via at least one interface. As used herein, the term “liquid chromatography (LC) unit”, in an embodiment, relates to an analytical module configured to separate one or more analytes of interest of a sample from other components of the sample via liquid chromatography, in an embodiment for detection of the one or more analytes with the mass spectrometry device. The LC may be based on any separation principle deemed appropriate by the skilled person; in an embodiment, the LC is reverse phase chromatography, hydrophobic interaction chromatography, ion exchange chromatography, size exclusion chromatography, affinity chromatography, or chiral chromatography; in a further embodiment, the LC is reverse phase chromatography. The LC device may comprise at least one LC column. For example, the LC device may be a single-column LC device or a multi-column LC device having a plurality of LC columns. The LC column may have a stationary phase through which a mobile phase is pumped in order to separate and/or elute and/or transfer the analyte(s) of interest. The LC unit may be or may comprise at least one high-performance liquid chromatography (HPLC) unit and/or at least one micro liquid chromatography (uLC) device. The term “gas chromatography” is understood by the skilled person; in an embodiment, the same separation principles as for LC are applicable, however, the mobile phase being a gas in GC.

The terms “mixing” and “admixing” are understood by the skilled person and are used interchangeably herein. In an embodiment, the terms include any and all measures deemed appropriate by the skilled person and causing the indicated components and optional further components to become mixed, in an embodiment to homogeneity. Unless otherwise indicated, the compounds can be added for mixing in any arbitrary order. Also, components may be added in a premixed form.

The term “isotopologue” is used herein in its conventional meaning to relate to a compound or group of compounds differing from a comparison compound only in its or their isotopic composition. Thus, the ions determined for the isotopologues in the method in an embodiment differ only in their isotope composition, in a further embodiment, the ions determined for the isotopologues and the analyte differ only in their isotope composition. As the skilled person is aware of, stable isomers, and therefore isotopologues, may be present in a preparation of a chemical compound in a statistical manner, caused e.g. by the natural distribution of isotopes. E.g. any carbon atom in a chemical molecule has a probability of about 1% of being ¹³C, unless artificially enriched or depleted. Thus, also in concurrence with conventional use, the term isotopologue also includes a population of molecules having the same isotope composition, but differing in the specific position of the isotope label (isotopomers). Thus, e.g. the ¹³C₁-isotopologue of testosterone includes 1-¹³C₁-testosterone, 2-¹³C₁-testosterone, 3-¹³C₁-testosterone, 4-¹³C₁-testosterone, etc. In view of the above, in an embodiment, the isotopologue is structurally identical to the analyte, but comprises at least one isotope label, i.e. in an embodiment at least one position in which an atom is replaced by an isotope thereof. For the avoidance of doubt, as used herein, the term isotopologue refers a population of molecules in which, compared to an analyte, the same number of atoms was replaced by the same isotope; this may be a population of molecules in which the same atom(s) in the same positions(s) were replaced, in which case the isotopologue may also be referred to as “specific isotopologue”; the isotopologue may, however, also be population of molecules in which the same number of the same atoms was replaced by the same number of the same isotopes, in which case the isotopologue may also be referred to as “mixed isotopologue”.

In an embodiment, the isotope is a stable isotope. In an embodiment, the atom replaced is a carbon atom and the isotope is ¹³C; in a further embodiment, the atom replaced is nitrogen and the isotope is ¹⁵N; in a further embodiment, the atom replaced is hydrogen and the isotope is 2 H; in a further embodiment, the atom replaced is oxygen and the isotope is ¹⁷O or ¹⁸O, in an embodiment is ¹⁸O; in a further embodiment, the atom replaced is sulfur and the isotope is ³³S or ³⁴S. In an embodiment, at least two, in a further embodiment at least three, in a further embodiment at least four, in a further embodiment at least five atoms in the analyte structure are replaced by their isotopes; in such case, in an embodiment only atoms of the same element are replaced and the isotopes replacing the atoms are the same isotopes. Thus, in an embodiment, in case carbon atoms are replaced, all carbon atoms replaced are replaced by 13 C atoms. In a further embodiment, at least 10%, in a further embodiment at least 20% in a further embodiment at least 30%, of the atoms of a specific element are replaced by a specific isotope. As the skilled person will understand, the above applies mutatis mutandis to fragments of the analyte and of the isotopologues which may be generated and detected during MS.

The term “internal calibrator”, as used herein, relates to a composition of matter comprising at least two non-identical isotopologues of an analyte at predetermined amounts. As will be understood by the skilled person in view of the instant description, the compounds being non-identical isotopologues of an analyte are in an embodiment mutually are isotopologues as well. Thus, in an embodiment, the non-identical isotopologues mutually are not isotopomers and/or are not isotopomers of the analyte.

In an embodiment, the internal calibrator comprises at least a first and a second isotopologue; in a further embodiment further comprises at least a third, in a further embodiment at least a fourth isotopologue of the analyte. The designations of the isotopologues are, in an embodiment, ordered by their relative amount in the internal calibrator, i.e. in an embodiment, the first isotopologue is the isotopologue present with the highest fraction, the second isotopologue is the isotopologue present with the second-highest fraction, and so forth. In an embodiment, the internal calibrator comprises at least a first isotopologue of the analyte as the main compound and at least a second isotopologue at a relative fraction of at most 20%, in an embodiment at most 10%, in a further embodiment at most 5% of the first isotopologue. In an embodiment, the at least two isotopologues or ions derived therefrom have m/z ratios in the range x to x+(n−1), with x being the m/z ratio of the isotopologue with the lowest m/z ratio and n being the number of isotopologues present in the internal calibrator. Thus, in an embodiment, the internal calibrator provides a series of ions with m/z values increasing by one mas unit each.

In an embodiment, the first isotopologue in the internal calibrator comprises or is an isotopologue of the analyte being labeled at specific positions within the molecule, in an embodiment at at least two, in a further embodiment at least three, in a further embodiment at least four positions. Thus, in an embodiment, the first isotopologue is a two-isotope, in a further embodiment a three-isotope, in a further embodiment a four-isotope, derivative of the analyte. Thus, the first isotopologue, in an embodiment, is a specific isotopologue as specified herein above.

In an embodiment, the second isotopologue is present in a preparation of the first isotopologue as a result of natural isotope distribution. In a further embodiment, a second and a third, in a further embodiment a second, a third, and a fourth isotopologue are present in a preparation of the first isotopologue as a result of natural isotope distribution. Thus, in an embodiment, the second isotopologue and optional third and further isotopologues is/are (a) mixed isotopologue(s) as specified herein above. Thus, in an embodiment, all isotopologues of the internal calibrator are comprised in a single isotopically labelled preparation of the analyte. Since the natural distribution of isotopes of an element is known, it is in principle possible to calculate the fractions of the second and further isotopologues from the probability that an atom is an isotope. In an embodiment, the relative fractions of the isotopologues in the internal calibrator are determined, e.g. by MS, or are provided by the manufacturer.

In an exemplary embodiment, the analyte may be Testosterone; in such case, the first isotopologue may be e.g. 2,3,4-¹³C₃-Testosterone; thus, the first isotopologue is the isotopologue with three carbon atoms at positions 2, 3, and 4 replaced by 13 C, thus having an m/z value increased by +3 compared to Testosterone. Further, Testosterone has 19 carbon atoms, so of the 16 formally unlabeled carbon atoms of 2,3,4-¹³C₃-Testosterone, each has a probability of about 1% of being 13 C, so about 16% of the 2,3,4-¹³C₃-Testosterone preparation will comprise one additional 13 C atom, and about 2.4% will comprise two additional ¹³C atoms.

In an embodiment, the second isotopologue comprises or is an isotopologue of the analyte being labeled at one additional or fewer position within the molecule compared to the first isotopologue. In an embodiment, the aforesaid applies to optional third and further isotopologues mutatis mutandis; in accordance, the second and optional third and further isotopologues is/are (a) specific isotopologue(s) as specified herein above Thus, in case the first isotopologue is 3,4-¹³C₂-testosterone, the second isotopologue could e.g. be 2,3,4-¹³C₃-Testosterone. In the case that the second isotopologue is an isotopologue labelled at one or more specific atoms in the molecule, the skilled person takes into account that, depending on natural isotope distribution, the preparation of the first isotopologue may comprise significant amounts of second isotopologue.

In view of the above, the relative fraction of the second isotopologue in an embodiment is a consequence of relative isotope distribution in the production process of the internal calibrator, and said relative isotope distribution may be a natural relative isotope distribution or an enriched relative isotope distribution.

As indicated above, the internal calibrator comprises at least two isotopologues of the analyte at predetermined amounts. The term “predetermined amount”, as used herein, relates to an amount which is fixed and documented at least before the calibration of the instant method is performed. The pre-determined amount of an internal calibrator may relate to any parameter correlating with the amount of internal calibrator added to a sample, in an embodiment is a volume of an internal calibrator stock solution. As the skilled person will understand, the predetermined amount as such does not have necessarily to be known at the time of admixing the internal calibrator to the sample, but it will be required to be known at step (c) of the method. As also indicated herein above, the predetermined amounts of isotopologues may, in principle, be calculated amounts, i.e. the predetermined amount of internal calibrator may be calculated from the weight of internal calibrator compound used, the volume it was dissolved in, and the volume of the resulting solution admixed to the sample. Also, the pre-determined amounts of isotopologues may be calculated as indicated herein above. In an embodiment, however, the pre-determined amounts of the isotopologues in the internal calibrator compound are determined by measurement, e.g. by determining MS signals of the internal calibrator. Also, the pre-determined amount of internal calibrator may be determined by measuring the concentration of the isotopologues in the internal calibrator by standard methods known to the skilled person, such as quantitative NMR (Q-NMR), GC, LC, and/or spectroscopic or immunologic methods.

The term “MS signal” is used herein in its customary meaning known to the skilled person. In an embodiment, the term relates to any intensity parameter or value thereof detected by the detector of an MS device for a specific m/z value or for a range of m/z values. The MS signal determined at an m/z value or range of m/z values of ions generated from the analyte is related to herein as “analyte signal”, and an MS signal determined at an m/z value or range of m/z values of ions generated from an isotopologue is related to herein as “isotopologue signal”.

The term “determining an MS signal” is understood by the skilled person as well. Determining MS signals according to the method specified herein comprises determining MS signals of ions generated from the analyte (analyte signal) and from said at least two isotopologues (isotopologue signals). The ions generated and detected will, as the skilled person understands, depend on the analyte, in particular its molecular weight and structure, as well as on the ionization method applied and the specific mode of MS detection envisaged. In an embodiment, the MS detection comprises detection in MS mode, in MS/MS mode, or any mode deemed appropriate by the skilled person, in particular as appropriate for the MS device as specified elsewhere herein.

In an embodiment, the term relates to determining a correlation plot of an MS signal with the m/z value or range of m/z values of the ions causing said signal. A graphical representation of a mass spectrum may be provided e.g. as a centroid graph and/or as a continuum graph. As referred to herein, the analyte signal and the isotopologue signal may be determined as separate signals; an isotopologue signal may, however also be determined as a combined signal; e.g. a signal of a first isotopologue (first isotopologue signal) may be determined as part of a determination of a combined analyte signal+first isotopologue signal, a signal of a second isotopologue (first isotopologue signal) may be determined as part of a determination of a combined analyte signal+second isotopologue signal, and so forth. Thus, the isotopologue does not necessarily have to be determined in an isolated manner. However the analyte signal will in an embodiment be determined as an isolated analyte signal.

The term “calibration” is used herein in a broad sense in concurrence with typical use by the skilled person. Thus, the term calibration includes an operation which establishes under specified conditions a relation between quantity values obtained with measurement standards and corresponding quantity values of a calibrated instrument, i.e. a calibration sensu stricto. Calibration may, however, also be verification of measurement values. The term calibration further includes measures of adjusting or re-adjusting the calibrated instrument or its output to concur with the aforesaid quantity values obtained with measurement standards comprised in the internal calibrator, i.e. calibration in its usual, broader sense. Thus, in the method specified herein, the term calibration may also relate to the provision of a correlation of MS signals determined by an MS device with amounts of isotopologues comprised in a sample.

According the step (a) of the method of determining an analyte, a pre-determined amount of internal calibrator is a admixed to said sample, wherein said internal calibrator comprises at least two non-identical isotopologues of the analyte at pre-determined amounts. Thus, in, i.e. before, during, and/or after, the sample preparation step, the internal calibrator is admixed to the sample. The amount of the internal calibrator is pre-determined, as specified herein above.

According to step (b) of the method of determining an analyte, MS signals of ions generated from the analyte (analyte signal) and from the at least two isotopologues (isotopologue signals) are determined. In an embodiment, the signal of the analyte is determined at the m/z value of the main ion generated from the analyte, i.e. the ion with the highest abundance, such as the M+H ion. In case the signal of the main ion is too high, other ions of lower abundance may be used as well. In case the isotopologue in the internal calibrator with the lowest molecular mass is removed by more than one, in an embodiment more than two mass units, the minor ion may as well be the +1 isotopologue of the analyte. In an embodiment, determining MS signals of ions in step (b) comprises determining MS signals of ions which are non-identical isotopomers between the analyte and the at least two non-identical isotopologues thereof. In a further embodiment, determining MS signals of ions in step (b) comprises determining MS signals of ions of said at least two non-identical isotopologues differing by one mass unit, i.e. one m/z unit. As the skilled person understands, in an embodiment, in each case corresponding ions are determined, i.e. in case a fragment ion of the analyte is determined, ions of the isotopologues are determined which have the same structure. In step (b), the analyte signal and the isotopologue signal may be determined separately; however, as specified herein above, also combined analyte+isotopologue signals may be determined.

According to step (c) of the method of determining an analyte, a calibration based on the analyte signal and the isotopologue signals determined in step (b) is provided. Thus, the calibration of the method of determining an analyte makes use of the isotopologue signals as well as of the analyte signal. Step (c) may comprise a substep of calculating the amounts of the respective isotopologues in the sample from the pre-determined amount of internal calibrator added to the sample and the pre-determined amounts of the isotopologues in the internal calibrator of step (a). In an embodiment, the calibration comprises correlating the combined signals of the analyte and the first, the second, and optional further isotopologues, respectively, to the pre-determined amount of the respective isotopologue; i.e., in an embodiment, step (c) comprises providing a calibration based on ratios of (i) the sum of the isotopologue signal and the analyte signal for each of said at least two isotopologues (ii) and the amounts for each of said at least two isotopologues. So, in an embodiment, the analyte+first isotopologue signal is correlated with the amount of the first isotopologue, the analyte+second isotopologue signal is correlated with the amount of second isotopologue in the sample, and so forth. As indicated herein above, said sums may be calculated sums, or may be determined directly as such in step (b). In an embodiment, the aforesaid sums are calculated sums, i.e. are calculated as the sums of the analyte signal and the respective isotopologue signals determined separately in step (b) and said analyte signal is linear up to an amount of analyte corresponding to the sum of the amount of analyte and the amount of the isotopologue with the highest concentration in the internal calibrator. In an embodiment, the aforesaid correlation comprises providing ratios of the combined signals to the respective amounts of the isotopologues; said ration may be graphically represented by a graph of amount of isotopologue over combined signal. In a further embodiment, calibration further comprises fitting a regression equation into said ratios. Said regression may be of any type deemed appropriate by the skilled person, including linear, polynomial, exponential, or other regression equations. In an embodiment, the regression equation is a linear regression equation.

According to step (d) of the method of determining an analyte, the analyte is determined based on the calibration provided in step (c). As will be understood by the skilled person, since the calibration of step (c) is based already, among others, on the analyte signal, this calibration in an embodiment permits determining the analyte directly from the calibration. Thus, in particular in case the regression equation used in calibration is linear, the amount of the analyte is determined as the negative value of the solution of the regression equation for the value of the MS signal of ions being zero. In an embodiment, the aforesaid amount is the monoisotopic amount of the analyte, i.e. the amount of analyte represented by its isotopologue with the highest abundance and/or corresponding to the calibrator isotopologue ion with the the same m/z value. Thus, in an embodiment, the aforesaid amount is corrected for the natural abundance of isotopes within the analyte, which distribution is known to the skilled person; thus, in an embodiment, the aforesaid amount is corrected by adding the amount(s) of isotopologue(s) of the analyte predictably present in the sample.

Advantageously, it was found in the work underlying the present invention that multipoint internal standard regression may be combined with standard addition methods to provide for calibration in e.g. MS methods. By such combination, it is in an embodiment not required that the analyte concentration lies within the range of known concentrations used for calibration. Thus, an analyte may be determined at any concentration. Moreover, it was found that isotope-labeled preparations of analytes may comprise amounts of isotopomers usable for establishing a calibration, thus making it possible to provide a calibration curve by just adding one isotope-labeled preparation of an analyte. Moreover, the relative content of the isotope-labeled preparation of the analyte can be established on-the-fly, either by determining the MS signal of the isotope-labeled preparations of the analyte as such, or from the sample according to step (a).

The definitions made above apply mutatis mutandis to the following. Additional definitions and explanations made further below also apply for all embodiments described in this specification mutatis mutandis.

The present invention further relates to a device configured to perform at least step (c), in an embodiment steps (c) and (d), of the method for determining an analyte of the present invention.

The term “device”, as used herein, relates to a collection of means comprising at least the indicated means operatively linked to each other, in an embodiment to allow at least a calibration to be provided. Typical means for calibration are known to the skilled person and include in particular a data processing unit comprising, in an embodiment, a data processor having tangibly embedded an algorithm which performs the calibration. The aforesaid computer algorithm may, however, also be comprised in a memory unit of the device. Thus, the device in an embodiment comprises a microprocessor and a memory unit, and in a further embodiment the device comprises, in an embodiment tangibly embedded in the memory unit, an algorithm causing the device to perform at least step c) of the method of determining an analyte when executed by the microprocessor. In an embodiment, the device further comprises at least one data interface for receiving data, such as values of MS signals determined in step (b), pre-determined amounts of internal calibrator and/or of isotopologues of step (a), and/or for outputting data, e.g. regression equations, calibration graphs, and/or an amount of an analyte. The aforesaid output data may as well be output via an optional output unit, e.g. a screen, a printer, or the like. The results may be given as output of raw data which need interpretation by a technician. In an embodiment, the output of the device is, however, processed, i.e. evaluated, raw data, the interpretation of which does not require a technician. How to link the means of the device in an operating manner will depend on the type of means included into the device. The person skilled in the art will realize how to provide and link appropriate means without further ado. In an embodiment, the means are comprised by a single device.

The present invention also relates to a system comprising a device as specified herein above operatively connected to a mass spectrometry device.

The term “system”, as used herein, relates to a system of means comprising at least the indicated devices operatively linked to each other. The system comprises at least one device as specified herein above and at least one MS device as also specified herein above. The skilled person knows how to operatively connect the device to an MS device; in an embodiment, the operative linkage comprises at least one common data interface. In an embodiment, the system is adapted to perform the method of determining an analyte as specified herein. In an embodiment, the mass spectrometry unit is adapted to perform at least step (b), in an embodiment at least steps (a) and (b) of the method of determining an analyte, and/or the device is adapted to perform at least step (c), in an embodiment steps (c) and (d) of the method of determining an analyte.

The present invention further relates to a use of a composition comprising at least two non-identical isotopologues of an analyte at a predetermined ratio for the determination of said analyte in a sample, in an embodiment according to the method of determining an analyte specified herein above.

The invention further discloses and proposes a computer program including computer-executable instructions for performing the method according to the present invention in one or more of the embodiments enclosed herein when the program is executed on a computer or computer network. Specifically, the computer program may be stored on a computer-readable data carrier. Thus, specifically, one, more than one or even all of method steps a) to d) as indicated above may be performed by using a computer or a computer network, in an embodiment by using a computer program.

The invention further discloses and proposes a computer program product having program code means, in order to perform the method according to the present invention in one or more of the embodiments enclosed herein when the program is executed on a computer or computer network. Specifically, the program code means may be stored on a computer-readable data carrier.

Further, the invention discloses and proposes a data carrier having a data structure stored thereon, which, after loading into a computer or computer network, such as into a working memory or main memory of the computer or computer network, may execute the method according to one or more of the embodiments disclosed herein.

The invention further proposes and discloses a computer program product with program code means stored on a machine-readable carrier, in order to perform the method according to one or more of the embodiments disclosed herein, when the program is executed on a computer or computer network. As used herein, a computer program product refers to the program as a tradable product. The product may generally exist in an arbitrary format, such as in a paper format, or on a computer-readable data carrier. Specifically, the computer program product may be distributed over a data network.

Finally, the invention proposes and discloses a modulated data signal which contains instructions readable by a computer system or computer network, for performing the method according to one or more of the embodiments disclosed herein.

In an embodiment, referring to the computer-implemented aspects of the invention, one or more of the method steps or even all of the method steps of the method according to one or more of the embodiments disclosed herein may be performed by using a computer or computer network. Thus, generally, any of the method steps including provision and/or manipulation of data may be performed by using a computer or computer network. Generally, these method steps may include any of the method steps, typically except for method steps requiring manual work, such as providing the samples and/or certain aspects of performing the actual measurements.

Specifically, the present invention further discloses:
A computer or computer network comprising at least one processor, wherein the processor is adapted to perform the method according to one of the embodiments described in this description,
a computer loadable data structure that is adapted to perform the method according to one of the embodiments described in this description while the data structure is being executed on a computer,
a computer program, wherein the computer program is adapted to perform the method according to one of the embodiments described in this description while the program is being executed on a computer,
a computer program comprising program means for performing the method according to one of the embodiments described in this description while the computer program is being executed on a computer or on a computer network,
a computer program comprising program means according to the preceding embodiment, wherein the program means are stored on a storage medium readable to a computer,
a storage medium, wherein a data structure is stored on the storage medium and wherein the data structure is adapted to perform the method according to one of the embodiments described in this description after having been loaded into a main and/or working storage of a computer or of a computer network, and
a computer program product having program code means, wherein the program code means can be stored or are stored on a storage medium, for performing the method according to one of the embodiments described in this description, if the program code means are executed on a computer or on a computer network.

Summarizing the findings of the present invention, the following embodiments are particularly envisaged:

Embodiment 1: A method of determining an analyte in a sample by mass spectrometry (MS), the method comprising
(a) admixing a pre-determined amount of internal calibrator to said sample, wherein said internal calibrator comprises at least two non-identical isotopologues of the analyte at predetermined amounts;
(b) determining MS signals of ions generated from said analyte (analyte signal) and from said at least two isotopologues (isotopologue signals);
(c) providing a calibration based on the analyte signal and the isotopologue signals determined in step (b); and
(d) determining said analyte based on the calibration provided in step (c).

Embodiment 2: The method of embodiment 1, wherein said internal calibrator comprises a first isotopologue of the analyte as the main compound and at least a second isotopologue at a relative fraction of at most 20%, in an embodiment at most 10%, in a further embodiment at most 5% of the first isotopologue.

Embodiment 3: The method of embodiment 2, wherein the relative fraction of the second isotopologue is a consequence of relative isotope distribution in the production process of the internal calibrator.

Embodiment 4: The method of embodiment 3, wherein said relative isotope distribution is a natural relative isotope distribution or an enriched relative isotope distribution.

Embodiment 5: The method of any one of embodiments 1 to 4, wherein said isotopologues are comprised in a single isotopically labelled preparation of the analyte.

Embodiment 6: The method of any one of embodiments 1 to 5, wherein said analyte comprises at least 10, in an embodiment at least 15, atoms of the same element, in an embodiment carbon atoms.

Embodiment 7: The method of any one of embodiments 1 to 6, wherein step (c) comprises providing a calibration based on the MS signals determined in step (b) and the amounts of said at least two isotopologues.

Embodiment 8: The method of any one of embodiments 1 to 7, wherein step (c) comprises providing a calibration based on ratios of (i) the sum of the isotopologue signal and the analyte signal for each of said at least two isotopologues (ii) and the amounts for each of said at least two isotopologues.

Embodiment 9: The method of any one of embodiments 1 to 8, wherein said sum is measured directly in step (b).

Embodiment 10: The method of any one of embodiments 1 to 8, wherein said sum is a calculated sum and, in an embodiment, wherein said analyte signal is linear up to an amount of analyte corresponding to the sum of the amount of analyte and the amount of the isotopologue with the highest concentration in the internal calibrator.

Embodiment 11: The method of any one of embodiments 8 to 10, wherein a regression equation is fitted into said ratios, and wherein the amount of the analyte is determined as the negative value of the solution of the regression equation for the value of the MS signal of ions being zero.

Embodiment 12: The method of embodiment 11, wherein said regression equation is a linear regression equation.

Embodiment 13: The method of any one of embodiments 1 to 12, wherein two of said at least two isotopologues differ in molecular mass by one mass unit.

Embodiment 14: The method of any one of embodiments 1 to 13, wherein said at least two isotopologues or ions derived therefrom have m/z ratios in the range x to x+(n−1), with x being the m/z ratio of the isotopologue with the lowest m/z ratio and n being the number of isotopologues present in the internal calibrator.

Embodiment 15: The method of any one of embodiments 1 to 14, wherein said isotopologues mutually are not isotopomers.

Embodiment 16: The method of any one of embodiments 1 to 15, wherein the determining MS signals of ions in step (b) comprises determining MS signals of ions which are non-identical isotopomers between the analyte and the at least two non-identical isotopologues thereof.

Embodiment 17: The method of any one of embodiments 1 to 16, wherein the determining MS signals of ions in step (b) comprises determining MS signals of ions of said at least two non-identical isotopologues differing by one mass unit.

Embodiment 18: The method of any one of embodiments 1 to 17, wherein said at least two isotopologues are at least three, in an embodiment at least four, isotopologues.

Embodiment 19: The method of any one of embodiments 1 to 18, wherein the ions determined for the isotopologues, and in an embodiment for the analyte, are structurally identical.

Embodiment 20: The method of any one of embodiments 1 to 19, wherein the ions determined for the isotopologues differ only in their isotope composition.

Embodiment 21: The method of any one of embodiments 1 to 20, wherein the ions determined for the isotopologues and the analyte differ only in their isotope composition.

Embodiment 22: The method of any one of embodiments 1 to 21, wherein said sample is a complex matrix sample, in an embodiment comprising more than 100, in a further embodiment more than 1000, chemical compounds.

Embodiment 23: The method of any one of embodiments 1 to 21, wherein said sample is a sample of a subject, in an embodiment a medical sample.

Embodiment 24: The method of any one of embodiments 1 to 23, wherein said sample is a sample of a bodily fluid or of a tissue sample.

Embodiment 25: The method of any one of embodiments 1 to 24, wherein said sample is a serum, plasma, or blood sample.

Embodiment 26: The method of any one of embodiments 1 to 25, wherein said analyte is a low-molecular weight compound, in an embodiment having a molecular mass of at most 2.5 kDa, in an embodiment at most 1.5 kDa, in a further embodiment at most 1 kDa.

Embodiment 27: The method of any one of embodiments 1 to 26, wherein said analyte is selected from the list consisting of testosterone; amphetamine; cocaine; methadone; ethyl glucuronide; ethyl sulfate; an opiate; an opioid; clonazepam; methotrexate; voriconazole; mycophenolic acid (total); mycophenolic acid-glucuronide; acetaminophen; salicylic acid; theophylline; digoxin; an immuno suppressant drug; an analgesic; an antibiotic; an antieplileptic; a hormone; and a vitamin.

Embodiment 28: The method of any one of embodiments 1 to 27, wherein said analyte is Testosterone and wherein said internal calibrator is 2,3,4-¹³C₃-Testosterone.

Embodiment 29: The method of any one of embodiments 1 to 28, wherein at least step (c), in an embodiment steps (c) and (d), of said method is/are computer-implemented.

Embodiment 30: The method of any one of embodiments 1 to 29, wherein said method further comprises outputting an amount or concentration of the analyte in the sample based on the determining in step (d).

Embodiment 31: A device configured to perform at least step c), in an embodiment steps (c) and (d), of the method according to any one of embodiments 1 to 30.

Embodiment 32: The device of embodiment 31, wherein said device comprises a microprocessor and a memory unit.

Embodiment 33: The device of embodiment 32, wherein said device comprises, in an embodiment tangibly embedded in said memory unit, an algorithm causing the device to perform at least step c) of the method according to any one of embodiments 1 to 28 when executed by the microprocessor.

Embodiment 34: A system comprising a device according to any one of embodiments 31 to 33 operatively connected to a mass spectrometry device.

Embodiment 35: The system of embodiment 34, wherein said mass spectrometry device is adapted to perform at least step (b), in an embodiment at least steps (a) and (b) of the method according to any one of embodiments 1 to 30.

Embodiment 36: Use of a composition comprising at least two non-identical isotopologues of an analyte at a predetermined ratio for the determination of said analyte in a sample, in an embodiment according to the method according to any one of embodiments 1 to 30.

All references cited in this specification are herewith incorporated by reference with respect to their entire disclosure content and the disclosure content specifically mentioned in this specification.

Figure Legends

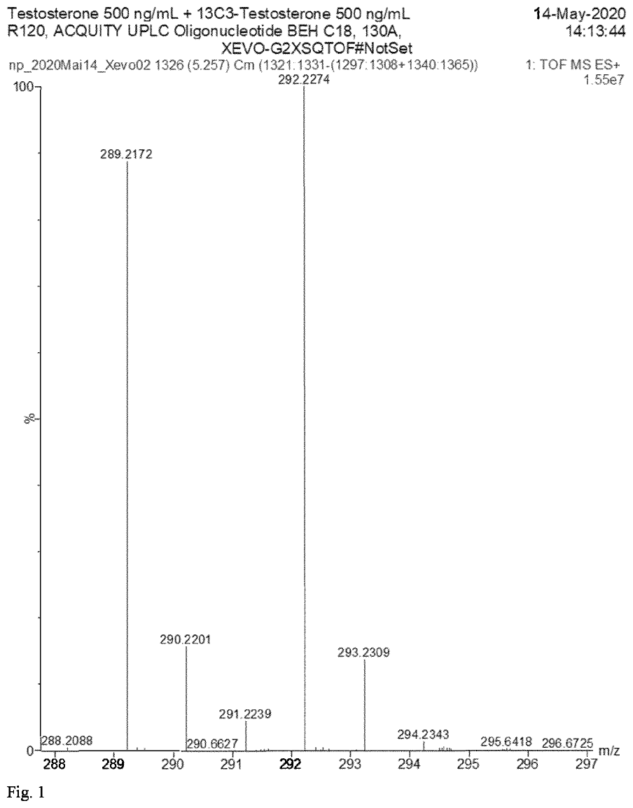

FIG. 1: Q-TOF MS spectrum of the artificial sample of Example 1.

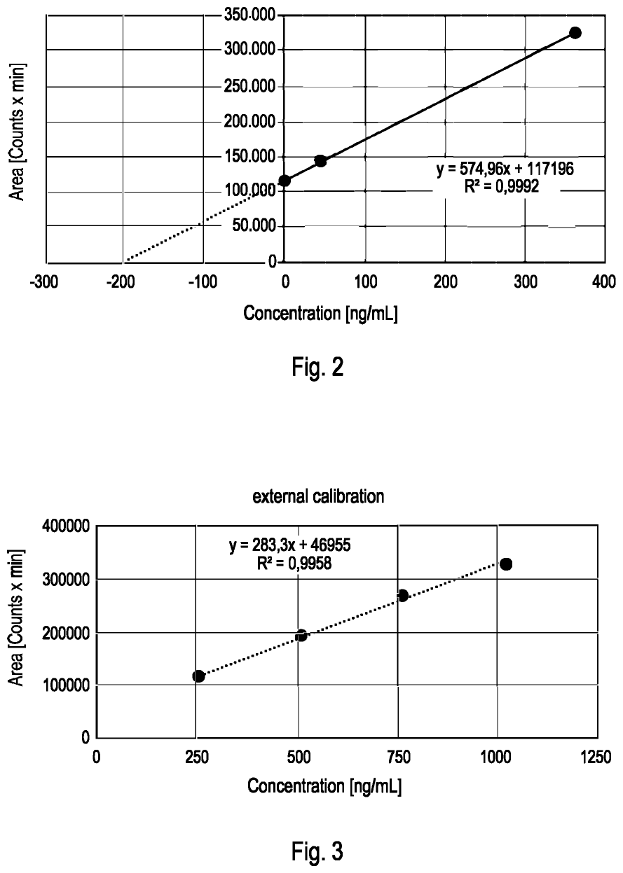

FIG. 2: Standard addition plot of the data of Table 3, Example 1.

FIG. 3. Calibration graph for external calibration (comparative Example 2).

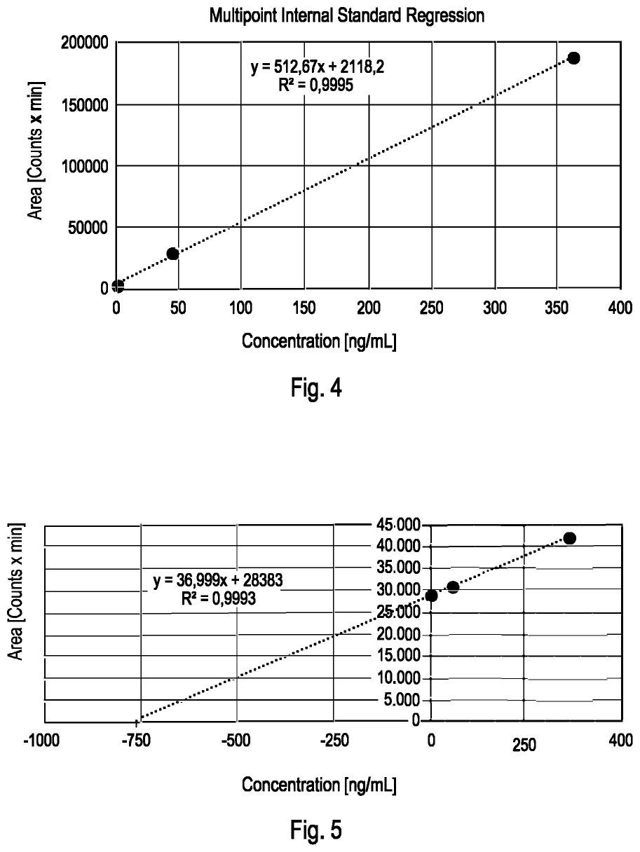

FIG. 4. Calibration graph for multipoint internal standard regression (comparative Example 3).

FIG. 5: Standard addition plot of the data of Example 4.

The following Examples shall merely illustrate the invention. They shall not be construed, whatsoever, to limit the scope of the invention.

### EXAMPLE 1: MS DETERMINATION OF TESTOSTERONE

1.1 Chemicals

The concentration of the Testosterone stock solution Cerilliant (1 mg/mL in Acetonitrile), Lot FE07241802, was determined by Q-PCR to be 1.018 mg/mL±2%. The concentration of the Testosterone stock solution 2,3,4-¹³C₃-Testosterone Cerilliant (0.1 mg/mL in Acetonitrile), Lot FE06071902. was determined by Q-PCR to be 0.083 mg/mL 5-7%.

Both stock solutions were diluted to use solutions having a concentration of 10 ng/mL with acetonitrile, and combined to an artificial sample comprising a nominal concentration of 255 ng/mL Testosterone and 415 n/mL 2,3,4-¹³C₃-Testosterone.

The content of the respective isotopologues in the artificial sample is shown in Tables 1 and 2:

TABLE 1
Isotopologues of Testosterone in the artificial sample.

| isotopologue [Da] | designation | fraction [%] | concentration [ng/mL] |
|---|---|---|---|
| 289.205 | [M + H] | 85.1 | 217.1 |
| 290.209 | [M + 1 + H] | 13.7 | 35.0 |
| 291.214 | [M + 2 + H] | 1.1 | 2.9 |

TABLE 2
Isotopologues of 2,3,4-¹³C₃-Testosterone in the artificial sample.

| isotopologue [Da] | designation | fraction [%] | concentration [ng/mL] |
|---|---|---|---|
| 291.212 | [M + 2 + H] | 1.2 | 4.8 |
| 292.215 | [M + 3 + H] | 87.2 | 362.0 |
| 293.219 | [M + 4 + H] | 10.9 | 45.0 |
| 294.224 | [M + 5 + H] | 0.8 | 3.2 |

The artificial sample was measured in an Q-TOF MS system. The resulting mass spectrum is shown in FIG. 1.

The respective ion areas (signal intensities) were measured and use to calculate data for a standard addition plot (Table 3), and to establish a standard addition plot (FIG. 2).

TABLE 3
MS signals of the artificial sample an sum values derived therefrom.
area [counts × min]

| | [M + 3 + | [M + 4 + | [M + 5 + | |
|---|---|---|---|---|
| calibrator isotopologue | H] | H] | H] | — |
| 2,3,4-¹³C₃-Testosterone | 209,565 | 32,037 | 2,029 | — |
| Testosterone [M + H] | 115,249 | 115,249 | 115,249 | 115,249 |
| area sum | 324,814 | 147,286 | 117,278 | 115,249 |
| concentration of | 362 | 45 | 3 | 0 |
| calibrator isotope | | | | |
| [ng/mL] | | | | |

From the calibration plot of FIG. 2 and the corresponding correlation equation y=574.96x+117,196, the concentration of the analyte Testosterone was determined to be 204 ng/mL, which compares to a nominal concentration of 217 ng/mL. This corresponds to a %-drift error of 6.

The same proceeding as above was tested for Testosterone concentrations of from 100 ng/mL to 1000 ng/mL, and it was found that lower concentrations tend to result in higher %-drift errors.

Compared to the comparative Examples below, the method of the invention has a lower %-drift error. Moreover, the method compensates for saturation effects and is highly flexible in that additional isotopologues can easily be added as calibrators.

### EXAMPLE 2: COMPARATIVE EXAMPLE: EXTERNAL CALIBRATION

Various Testosterone concentrations were determined in external calibration with Q-TOF MS; the measured signals are shown in Table 4, the calibration graph is shown in FIG. 3.

TABLE 4
Testosterone concentrations and measured signals.

| Testosterone concentration [ng/mL] | area [counts × min] |
|---|---|
| 250 | 115,249 |
| 500 | 193,048 |
| 750 | 270,872 |
| 1000 | 329,637 |
| test sample | 187,413 |

The resulting correlation equation was y=283.3x+46,955, which results in a determined concentration of 496 ng/mL in the test sample, which compares to a nominal concentration of 415 ng/mL; the 5-drift error was −19.

### EXAMPLE 3: COMPARATIVE EXAMPLE: MULTIPOINT INTERNAL STANDARD REGRESSION

The 2,3,4-¹³C₃-Testosterone preparation of Example 1 was also used in a multipoint internal standard regression calibration method. The concentrations of the standards and corresponding signals are shown in Table 5, the calibration graph is shown in FIG. 4.

TABLE 5
isotopologues and associated signals.

| | | concentration | area |
|---|---|---|---|
| isotopologue | designation | [ng/mL] | [counts × min] |
| 292.215 | [M + 3 + H] | 362.0 | 187,413 |
| 293.219 | [M + 4 + H] | 45.0 | 27,613 |
| 294.224 | [M + 5 + H] | 3.2 | 1,619 |

The resulting correlation equation was y=512.67x+2118.2, which results in a determined concentration of 372 ng/mL in the test sample, which compares to a nominal concentration of 433 ng/mL; the %-drift error was 14.

### EXAMPLE 4: DETERMINATION OF ESTRADIOL

The method was performed essentially as in Example 1, using estradiol as the analyte and ¹³C₅-estradiol as an internal calibrator on a Q-TOF MS device. The resulting calibration graph is shown in FIG. 5.

The %-drift error in the determination was about 11, which compares to 15 for external calibration (performed analogous to Example 2) and 14 in multipoint internal regression (performed analogous to Example 3).

### REFERENCES

Hoffman et al. (2020), Clin Chem 66(3):474
WO 2017/178453

1. A method of determining an analyte in a sample by mass spectrometry (MS), the method comprising
(a) admixing a pre-determined amount of internal calibrator to said sample, wherein said internal calibrator comprises at least two non-identical isotopologues of the analyte at predetermined amounts;
(b) determining MS signals of ions generated from said analyte (analyte signal) and from said at least two isotopologues (isotopologue signals);
(c) providing a calibration based on the analyte signal and the isotopologue signals determined in step (b); and
(d) determining said analyte based on the calibration provided in step (c).

2. The method of claim 1, wherein step (c) comprises providing a calibration based on ratios of (i) the sum of the isotopologue signal and the analyte signal for each of said at least two isotopologues (ii) and the amounts for each of said at least two isotopologues.

3. The method of claim 1, wherein a regression equation is fitted into said ratios, and wherein the amount of the analyte is determined as the negative value of the solution of the regression equation for the value of the MS signal of ions being zero.

4. The method of claim 3, wherein said regression equation is a linear regression equation.

5. The method of claim 1, wherein said internal calibrator comprises a first isotopologue of the analyte as the main compound and at least a second isotopologue at a relative fraction of at most 20%.

6. The method of claim 5, wherein the relative fraction of the second isotopologue is a consequence of relative isotope distribution in the production process of the internal calibrator.

7. The method of claim 1, wherein said isotopologues are comprised in a single isotopically labelled preparation of the analyte.

8. The method of claim 1, wherein the ions determined for the isotopologues are structurally identical.

9. The method of claim 1, wherein said sample is a sample of a subject.

10. The method of claim 1, wherein said analyte is a low-molecular weight compound.

11. The method of claim 1, wherein said analyte is Testosterone and wherein said internal calibrator is 2,3,4-¹³C₃-Testosterone.

12. A device configured to perform at least step c) of the method according to claim 1.

13. A system comprising a device according to claim 12 operatively connected to a mass spectrometry device.

14. (canceled)

15. (canceled)

16. The method of claim 8, wherein the ions determined for the analyte are structurally identical.

17. The method of claim 9, wherein said sample is a medical or diagnostic sample.

18. The method of claim 9, wherein said sample is a sample of a bodily fluid or of a tissue sample.

19. The method of claim 10, wherein said analyte is a low-molecular weight compound having a molecular mass of at most 2.5 kDa.

20. A device configured to perform at least steps (c) and (d) of the method according to claim 1.

21. The method of claim 5, wherein said internal calibrator comprises a first isotopologue of the analyte as the main compound and at least a second isotopologue at a relative fraction of at most 10% of the first isotopologue.

22. The method of claim 5, wherein said internal calibrator comprises a first isotopologue of the analyte as the main compound and at least a second isotopologue at a relative fraction of at most 5% of the first isotopologue.
